(19) United States
(12) Patent Application Publication
(10) Pub. No.: US 2006/0253203 A1
(43) Pub. Date: Nov. 9, 2006

(54) Hernial prosthesis for intraprosthetic fixation

(76) Inventor: Alfredo Alvarado, Weston, FL (US)

Correspondence Address: Mark D. Bowen;Stearns Weaver Miller, et al., Suite 1900, 200 East Broward Boulevard, Fort Lauderdale, FL, 33301, US

(21) Appl. No.: 11/121,244

(22) Filed: May 3, 2005

(51) Int. Cl. A61F 2/02

(52) U.S. Cl. 623/23.74; 606/151

(57) ABSTRACT

An implantable hernial prosthesis having top and bottom layers and a central sleeve to facilitate manual expansion and placement of the prosthesis within an incision in a patient. The top layer and a bottom layer are secured together with at least one seam at the perimeter of the prosthesis. The top layer is made of a synthetic mesh, preferably polypropylene mesh, to promote incorporation into the abdominal wall, and the bottom layer is made of the same material or in some cases of a mechanical barrier to prevent adhesions to the intestine. The top layer is provided with a central sleeve to introduce one or two fingers to expand the prosthesis in place, and also to introduce an articulated hernial stapler to secure the mesh into the abdominal wall.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights rights whatsoever.

### BACKGROUND OF THE INVENTION

1. Field of the Invention

The present invention relates to a new hernial prosthesis for use in surgical repair of abdominal wall hernias.

2. Description of Related Art

Surgically implanted mesh prosthesis have been used over the years for repair of abdominal wall hernias especially for the repair of incisional ventral hernias. This repair can be done by an open external technique or by an internal technique using laparoscopic instruments. The open technique requires dissection of the hernial sac and adjacent tissues in order to insert and secure the prosthesis in place through the same incision made at the center of the hernial defect. In this case the patch can be inserted into the preperitoneal space behind the abdominal wall muscles, or intraperitoneally behind the peritoneal layer. In both techniques the prosthesis can be secured in place with full thickness transabdominal sutures or with staples or spiral tacks. However the insertion of sutures to fix the prosthesis to the abdominal wall is time consuming and requires special suture passers. Furthermore these sutures may produce persistent pain at the insertion sites. On the other hand the insertion of staples or spiral tacks using laparoscopic techniques are also difficult to perform and may not hold as well as the transabdominal sutures.

In the majority of the cases the implantable prosthesis consists of a composite patch made of a physical barrier to prevent formation of adhesions on the peritoneal side, and a knitted polypropylene monofilament mesh on the outer side to promote ingrowth and incorporation of the mesh into the abdominal wall such as Composix from Bard, Inc. and Sepramesh from Genzyme, Inc. A different type of prosthesis is made of expanded polytetrafluoroethylene with a smooth surface on one side, and a corrugated surface on the other side (Goretex Dual Mesh). The smooth side faces the intestine and serves as an adhesion barrier, while the corrugated surface is applied against the abdominal wall to promotes fixation via cellular and collagen ingrowth.

U.S. Pat. No. 5,916,225, issued to Kugel, discloses a hernia patch having a first layer of inert synthetic mesh material selectively sized and shaped to extend across and beyond a hernia, and a second layer of inert synthetic mesh material overlies the first layer to create a generally planar configuration for the patch. The first and second layers are joined together by a seam which defines a periphery of a pouch between the layers. One of the layers has a border which extends beyond the seam and which has a free outer edge. An access slit is formed in one of the layers for insertion of a finger of a surgeon into the pouch to allow the surgeon to deform the planar configuration of the patch to facilitate insertion of the patch into the patient and to position the patch across the hernia.

U.S. Pat. No. 5,593,441, issued to Lichtenstein et al., discloses a composite prosthesis and method for limiting the incidence of postoperative adhesions. The composite includes a mesh fabric and a barrier which prevents exposure of the mesh fabric to areas of potential adhesion. The interstices of the mesh fabric are infiltrated by tissue which secures the prosthesis in place. The composite is positioned with the barrier relative to the region of potential adhesion, such as the abdominal viscera.

U.S. Pat. No. 5,147,374, issued to Fernandez, discloses a patch made from a rolled up first flat sheet of polypropylene or polytetrafluroethylene surgical mesh. One end of the rolled up mesh has multiple slits to provide multiple flared out flaps stitched to a second flat sheet of surgical mesh. The patch is compressed into a longitudinal cylindrical structure and is inserted through a trocar into an opening of a hernia. The rolled up first flat sheet is inserted through the opening and the flaps and second flat sheet are stapled to the patient's tissue adjacent the opening.

Other hernia repair devices are disclosed in U.S. Application Publication No. 2003/0181988 (Rousseau), 2003/0187516 (Amid et al.), U.S. Pat. No. 6,120,530 (Eldridge et al.), U.S. Pat. No. 4,769,038 (Bendavid et al.), U.S. Pat. Nos. 5,725,577 and 5,743,917 (Saxon).

The implantable prosthesis devices disclosed in the art are burdened by a number of disadvantages and have failed to gain widespread acceptance. Accordingly, there exists a need for an improved implantable prosthesis for use in the repair of abdominal wall hernias such as ventral incisional hernias and also inguinal and femoral hernias. There further exists a need for such a prosthesis wherein the dissection of the anatomical space needed for the insertion of the prosthesis can be done very easily by inserting one or two fingers through a central sleeve of the prosthesis. There further exists a need for an improved implantable prosthesis wherein fixation of the prosthesis can be accomplished by inserting an articulated hernia stapler, or in certain cases a spiral tacker, through the same central sleeve into the pocket formed between the two layers of the prosthesis.

### BRIEF SUMMARY OF THE INVENTION

The present invention overcomes the limitations and disadvantages in the art by providing an implantable hernial prosthesis having top and bottom layers and a central sleeve to facilitate manual expansion and placement of the prosthesis. The top layer and a bottom layer are secured together with only one seam at the perimeter of the prosthesis. The top layer is made of a synthetic mesh, preferably polypropylene mesh, to promote incorporation into the abdominal wall, and the bottom layer is made of the same material or in some cases of a mechanical barrier to prevent adhesions to the intestine. The top layer is provided with a central sleeve to introduce one or two fingers to expand the prosthesis in place, and also to introduce an articulated hernial stapler to secure the mesh into the abdominal wall.

Accordingly, it is an object of the present invention to provide an improved implantable hernial prosthesis.

Another object of the present invention is to provide a hernial prosthesis adapted such that the fixing instruments can be guided to the edges of the prosthesis in a safe manner by feeling the instrument with the fingers inserted inside the pocket.

Still another object of the present invention is to provide a hernail prosthesis wherein the staples or tacks are always covered by one layer of the prosthesis.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

FIG. 1A is a top view of a preferred embodiment hernial prosthesis in accordance with the present invention;

FIG. 1B is a side view thereof;

FIG. 2A is a top view of an alternate embodiment hernial prosthesis in accordance with the present invention;

FIG. 2B is a side view thereof;

FIGS. 3A-3C illustrate preperitoneal insertion of a hernial prosthesis of the instant invention;

FIG. 4A illustrates digital spreading of the inserted hernial prosthesis; and

FIG. 4B illustrates intraprosthetic fixation of the inserted hernial prosthesis using a articulated hernia stapler.

### DETAILED DESCRIPTION OF THE INVENTION

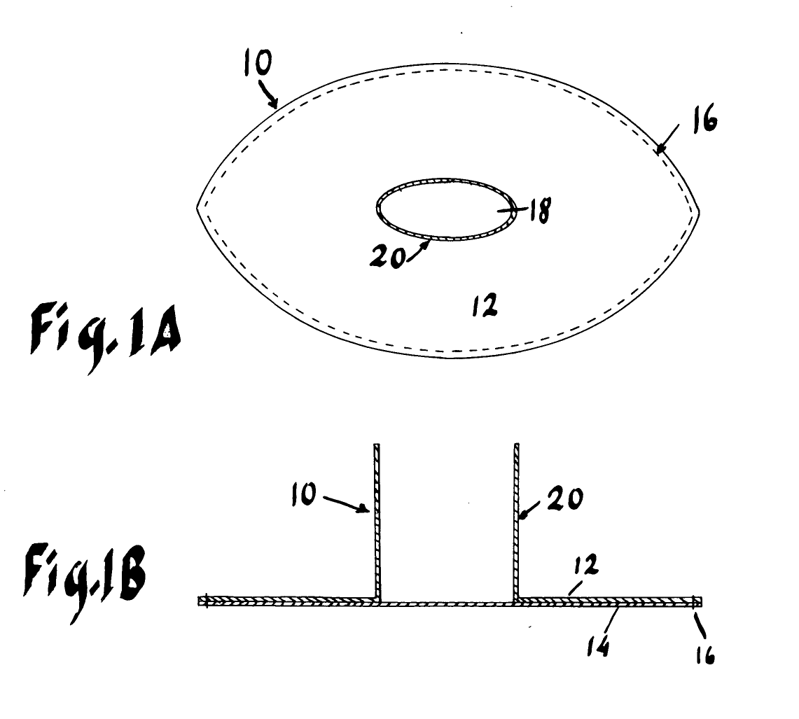

With reference now to the drawings, FIGS. 1A and 1B depict a preferred embodiment of a herinal prosthesis, generally referenced as 10, in accordance with the present invention.

Prosthesis 10 includes a top layer 12 and a bottom layer 14 secured together proximal the peripheral edges thereof by a seam 16. Seam 16 is preferably formed by stitching using a dark colored thread which contrasts with the lighter colored top and bottom layers to allow the surgeon to visually detect the peripheral edge of prosthesis 10 during surgery. In alternate embodiments, seam 16 may be formed using an adhesive, by fusing or welding the top and bottom layers, or by any other suitable method. Top and bottom layers 12 and 14 are prefereably fabricated from a synthetic mesh, such as polypropylene mesh, and is designed for extraperitoneal insertion of the mesh such as in the repair of inguinal or femoral hernias. Top layer 12 defines an opening 18, and includes a central sleeve 20 attached to top layer 12 in surrounding relation with opening 18 to allow for introduction of one or two fingers inside the prosthesis in order to expand the mesh in the selected anatomical space, and subsequently to introduce an articulated hernia stapler to secure the mesh into the abdominal wall as further discussed herein. Opening 18 is preferably oval-shape, but may be circular or any other suitable shape. Sleeve 20 may be formed as an integral part of top layer 12, or alternatively may be formed separately and connected to top layer 12 by suitable means of attachement. The size of sleeve 20 is preferably proportional to the size of the prosthesis. A significant advantage provided by central sleeve 20 is that the sleeve keeps the prosthesis at the center of the hernial defect during the procedure. Seam 16 is preferably fabricated using a black thread such that the surgeon may easily visualize the periphery of the mesh. At the end of the procedure sleeve 20 can be trimmed off near its base 20A and the resulting linear defect can be closed with a running suture.

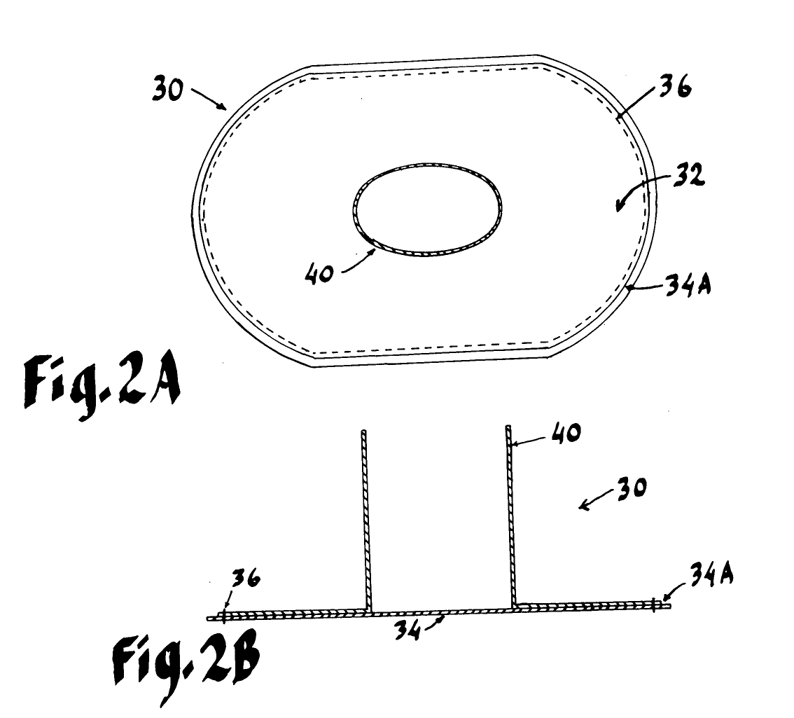

FIGS. 2A and 2B depict an alternate embodiment hernial prosthesis in accordance with the present invention, generally referenced as 30. Posthesis 30 includes a top layer 32 and a bottom layer 34 secured together by a seam 36. In this embodiment, bottom layer 34 is fabricated in the form of a generally solid (non-mesh) sheet of expanded polytetrafluoroethylene (ePTFE) or similar material to prevent adhesions to the intestine and it is designed for intraperitoneal insertion of the prosthesis. In this embodiment, bottom layer 34 is preferably slightly larger than top layer 32 such that the peripheral edge of bottom layer 34 extends beyond top layer 32 thereby forming a rim, referenced as 34A. Top layer 32 defines an opening 38, and includes a central sleeve 40 attached to top layer 32 in surrounding relation with opening 38 to allow for introduction of one or two fingers inside the prosthesis in order to expand the mesh in the selected anatomical space, and subsequently to introduce an articulated hernia stapler to secure the mesh into the abdominal wall as further discussed herein. The size of sleeve 40 is preferably proportional to the size of the prosthesis. A significant advantage provided by central sleeve 60 is that the sleeve keeps the prosthesis at the center of the hernial defect during the procedure. As disclosed above, seam 36 is preferably fabricated using a black thread, or other dark colored thread (i.e. purple), such that the surgeon may easily visualize the periphery of prosthesis 10. At the end of the procedure sleeve 40 can be trimmed off near its base 40A and the resulting linear defect can be closed with a running suture.

### Method of Insertion

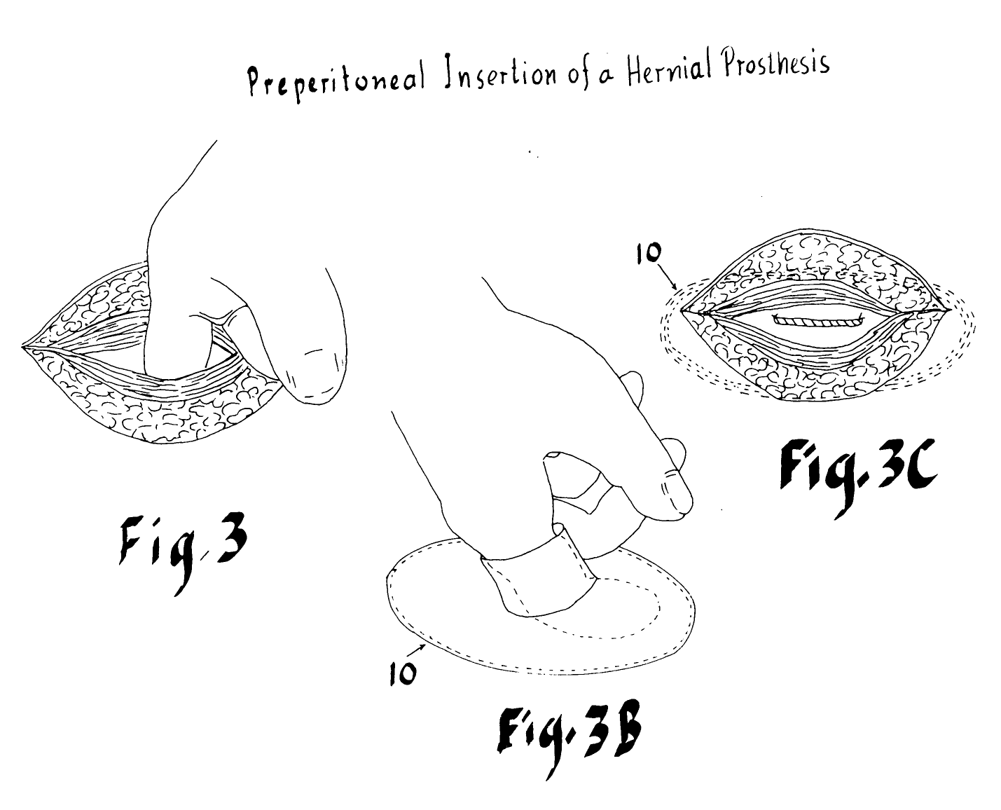

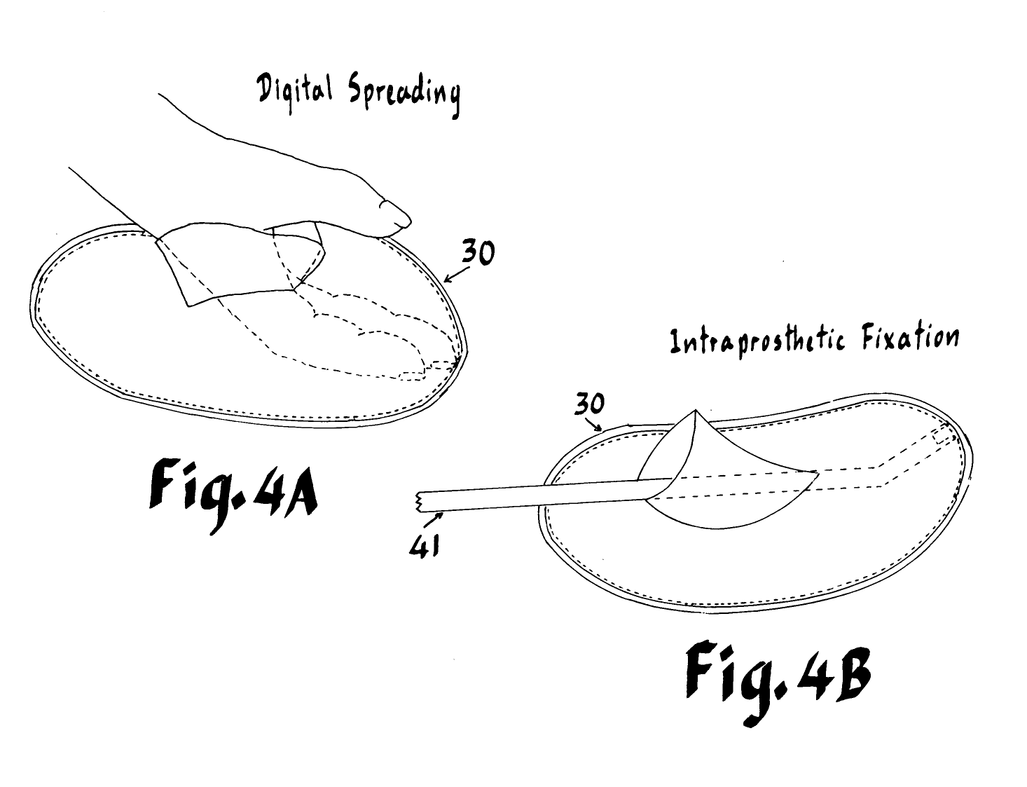

The hernial prosthesis of this invention can be inserted according to the preferred technique of the surgeon and the particular characteristics of the hernia under consideration. Turning now to FIGS. 3A-3C and 4A-4B, insertion of hernial prosthesis is disclosed. More particularly, in small ventral hernias and in inguinal and femoral hernias the preferred technique is to make a small incision at the center or near to the hernial defect and then to dissect the preperitoneal space to make room for the insertion of the prosthesis as illustrated in FIG. 3A. The prosthesis is spread with the index finger through the central sleeve as illustrated in FIG. 3B, and positioned with the central sleeve disposed through the incision site as illustrated in FIG. 3C. Finally, the prosthesis is secured in place with a few hernia staples using the articulated stapler or the helical stapler inside the prosthesis.

FIGS. 4A and 4B illustrate insertion of a large prosthesis in a large ventral hernia. In this application, the prosthesis can be inserted by the open technique at the site of the hernial defect. In this case the hernial sac is dissected out and excised at its neck to avoid and extensive dissection. Then the intraperitoneal space is cleared by pushing the bowel inside or by cutting intestinal adhesions as needed. The large prosthesis is inserted through the same incision into the abdomen, and spread with the fingers through the central sleeve followed by fixation with multiple staples using the articulated hernia stapler. Also large ventral hernias can be repaired using laparoscopic techniques in which the prosthesis is inserted through a small incision made at the center of the hernial defect following complete lysis of intraabdominal adhesions. Then the hernial prosthesis is fixed to the abdominal wall with numerous staples using the articulated hernia stapler inserted through the central sleeve. At the end of the procedure the sleeve is trimmed off with scissors and the linear defect is closed with a running suture of non-absorbable monofilament material.

The instant invention has been shown and described herein in what is considered to be the most practical and preferred embodiment. It is recognized, however, that departures may be made therefrom within the scope of the invention and that obvious modifications will occur to a person skilled in the art.

What I claim is:

1. A hernial prosthesis for intraprosthetic fixation within a patient, comprising:
a bottom layer of synthetic material sized and shaped for extraperitoneal insertion in covering relation with an incision on a patient;
a top layer of synthetic material having a peripheral edge attached to said bottom layer;
said top layer defining an opening connected to a sleeve sized to allow for insertion of at least one human finger.

2. A hernial prosthesis according to claim 1, wherein said bottom layer comprises polypropylene mesh.

3. A hernial prosthesis according to claim 1, wherein said bottom layer comprises generally solid sheet of expanded polytetrafluoroethylene.

4. A hernial prosthesis according to claim 1, wherein said top and bottom layers are connected by a peripheral stitched seam.

5. A hernial prosthesis according to claim 4, wherein said stitched seam is black.

6. A hernial prosthesis according to claim 4, wherein said bottom layer extends beyond said top layer along the entire peripheral edge.

7. An implantable hernial prosthesis for extraperitoneal insertion and intraprosthetic fixation within a patient, said hernial prosthesis comprising:
top and bottom layers of synthetic material connected generally along common peripheral edges thereof by a seam;
said top layer defining a generally centrally located opening;
said top layer further including a generally tubular sleeve of synthetic material connected to said top layer in surrounding relation with said opening for facilitating manual expansion and placement of the prosthesis by introduction of at least one finger of a surgeon though said tubular sleeve and said top layer opening.

8. A hernial prosthesis according to claim 7, wherein said bottom layer comprises polypropylene mesh.

9. A hernial prosthesis according to claim 7, wherein said bottom layer comprises generally solid sheet of expanded polytetrafluoroethylene.

10. A hernial prosthesis according to claim 7, wherein said peripheral seam is a stitched seam.

11. A hernial prosthesis according to claim 10, wherein said stitched seam is formed by dark thread.

12. A hernial prosthesis according to claim 7, wherein said bottom layer extends beyond said top layer along the entire peripheral edge.
